(19) United States
(12) Patent Application Publication
(10) Pub. No.: US 2024/0252567 A1
(43) Pub. Date: Aug. 1, 2024

(54) A WATER SOLUBLE OR WATER DISPERSIBLE BOLUS ARTICLE CONTAINING BROMOFORM

(71) Applicant: BIMEDA ANIMAL HEALTH LIMITED, Carrickmines, Dublin (IE)

(72) Inventors: Daniel Peter TIERNEY, Blackrock (IE); Donal Thomas Martin TIERNEY, Blessington (IE)

(73) Assignee: BIMEDA ANIMAL HEALTH LIMITED, Carrickmines, Dublin (IE)

(21) Appl. No.: 18/560,507

(22) PCT Filed: May 27, 2021
(86) PCT No.: PCT/EP2021/064290

§ 371 Date: Nov. 13, 2023

(30) Foreign Application Priority Data
May 14, 2021 (GB) 2106923.2

(51) Int. Cl. A61K 36/04; A61K 9/00; A61K 31/02; A61K 47/02; A61K 47/32

(52) U.S. Cl. CPC A61K 36/04; A61K 9/0053; A61K 31/02; A61K 47/02; A61K 47/32; A61K 2236/33; A61K 2236/35

(57) ABSTRACT

A water soluble or water dispersible bolus article suitable for oral administration to a ruminant animal comprising a seaweed extract enriched in bromoform is described. The bolus article is generally a rumen bolus article. The seaweed extract enriched in bromoform is a methanolic extract or an oil extract of seaweed. The bromoform constitutes at least 60% of the seaweed extract by weight. The seaweed extract is lyophilised seaweed extract. The seaweed extract is an extract of an Asparagopsis species of seaweed. A method of reducing methane production in ruminant animals is also described.

### FIELD OF THE INVENTION

The present invention relates to a water-soluble or water dispersible bolus article containing bromoform, a method of manufacturing a water soluble/dispersible bolus article, and use of the bromoform bolus article to reduce methane production in an animal especially a ruminant animal.

### BACKGROUND TO THE INVENTION

Dry seaweed has been shown to reduce methane production in beef steers (Roque et al. PLOS ONE 16(3): e0247820). In the study, 50-100 g of dry red seaweed was fed to cattle every day over a period of 5 months, resulting in reduction in methane production in the animals of between 63% and 88%. Halogenated CH₄ analogs such as chloroform and bromoform present in seaweed have been suggested as active agents capable of reducing methane production. Bromoform is present in dry red seaweed at 1723 ppm.

### SUMMARY OF THE INVENTION

The Applicant has realised that methane reduction in ruminant animals (cattle, sheep, goats) can be reduced by providing a bolus article that contains bromoform, where the bolus is configured for sustained release of a desired amount of bromoform over a predetermined period of time. The bolus article is particularly a rumen bolus article, suitable for administration to a reticulo rumen of a ruminant animal such as a bovine animal. The Applicant has also realised that providing a bolus article containing a seaweed extract that is enriched in bromoform, e.g. containing >60% bromoform by weight, allows a relatively large amount of bromoform to be formulated into a bolus, which enables an effective amount of bromoform (e.g. 100 mg) to be released daily from the bolus over a sustained period of time (e.g. up to 100 days) which would not be possible using dry seaweed. The invention therefore relates to a bolus article, especially a rumen bolus article, comprising bromoform, and in particular a seaweed extract enriched in bromoform (e.g. a methanolic extract or oil extract which is lyophilised). The article is water soluble or water dispersible to release the bromoform over a sustained period.

In any embodiment, bromoform constitutes at least 10%, 20%, 30%, 40%, 50% or 60% of the seaweed extract by weight.

In any embodiment, the bromoform (e.g. the seaweed extract enriched in bromoform) is lyophilised.

In any embodiment, the seaweed extract is lyophilised seaweed extract.

In any embodiment, the seaweed extract is an extract of an Asparagopsis species of seaweed.

In any embodiment, the bolus article comprises 1-50 g, 1-20 g, 5-15 g bromoform and is configured to release 10-200 mg 50-150 mg or 80-120 mg bromoform per day, especially in the reticulo rumen of a ruminant animal.

In any embodiment, the bolus article comprises a water soluble matrix and bromoform distributed throughout the matrix, in which the water soluble matrix comprises a water soluble glass.

In any embodiment, water soluble glass comprises a carboxylate glass.

In any embodiment, the carboxylate glass is formed from one or more metal carboxylates having a melting point of less than 300° C.

In any embodiment, water soluble glass comprises a polymer bonded glass.

The invention also provides a bolus article (especially a rumen bolus article) according to the invention, for use in a method of administering bromoform to a animal (especially a ruminant animal) by sustained release. The method generally comprises orally administering the bolus article to the stomach, or the reticulo rumen, of the animal wherein the bromoform is released from the bolus article in the animal over a sustained period of time.

In any embodiment, the bolus article comprises 5-15 g bromoform, and is configured to release 10-200 mg bromoform per day in the reticulo rumen of the animal for a sustained period of 20-100 days.

In any embodiment, the rumen bolus article comprises 5-15 g bromoform, and is configured to release 50-150 mg bromoform per day in the reticulo rumen of the animal for a sustained period of 20-90 days.

In any embodiment, the rumen bolus article comprises 5-15 g bromoform, and is configured to release 50-150 mg bromoform per day in the reticulo rumen of the animal for a sustained period of 30-60 days.

The invention also provides a bolus article (especially a rumen bolus article) according to invention, for use in a method of reducing methane production in an animal (especially a ruminant animal), the method comprising a step of administering a water-soluble glass bolus article to a stomach (for example the reticulo rumen) of the animal, wherein the bromoform is released from the bolus article in the animal over a sustained period of time.

Other aspects and preferred embodiments of the invention are defined and described in the other claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

“Bolus article”: Bolus articles are described in the literature and are used to deliver active agents over a sustained period of time to animals. The articles are implanted into the gastrointestinal track of the animal, generally the stomach of monogastric animals, but more usually into the reticulo rumen of a ruminant animal such as a goat, sheep or cow (e.g. a rumen bolus). The bolus article generally is formed with a water soluble or water dispersible matrix or shell that breaks up over time in the stomach or reticulo rumen of an animal releasing an active agent contained within the matrix over a sustained period. They are often employed for sustained release of micronutrients and other active agents. Water soluble glasses, waxes and other materials are used as matrix materials. Rumen bolus articles are described in the following documents: GB2163346, EP0042219, CN104523681, GB2037735 (all water soluble glass bolus articles), U.S. Pat. No. 5,720,972 (wax coat), U.S. Pat. No. 5,322,692 (barium sulphate) and GB2333451 (wax coat). In one embodiment, the bolus article is a glass bolus article. In one embodiment, the bolus article is a non-glass bolus article.

“Water soluble or water dispersible” means that the bolus article is configured to break down over time in the digestive tract (e.g. the reticulo rumen) of the animal by dissolving (e.g. water soluble glass bolus) or dispersing.

As used herein, the term “water-soluble glass bolus article” refers to a solid article suitable for administration to the stomach or reticulo rumen of an animal that comprises water soluble glass, in one embodiment carboxylate glass (or another low-temperature glass forming material). Metal carboxylates such as acetates, propanoates and butanoates can be melted to a glass and have a lower melting temperature that phosphate-based glass materials, allowing thermally sensitive active agents such as bromoform to be formulated into a glass bolus. The metal carboxylate (or mixture of metal carboxylates) employed generally have a melting point of less than 300° C., 250° C. or 200° C. In one embodiment, the glass if formed without the bromoform, and then milled into particles and the bromoform and optionally a binder is added, and the mixture is formed into a suitable article (generally by pressing) and the article is then sintered to form the glass bolus article. In any embodiment, the sintered article comprises a binder. In another embodiment, the glass forming carboxylate(s) are mixed with the bromoform, melted to a liquid glass (typically at a temperature of less than 149° C.), and then cast into a glass bolus article. In any embodiment, the article is sintered.

In any embodiment, the bolus article has a weight of 10 to 200 g, 50 to 200 g, 10 to 50 g, 50 to 150 g, 75 to 125 g, and ideally about 90 to 110 g. In any embodiment, the bolus article contains 1-20%, 1-5%, 5-15%, 5-10%, 10-15%, 15-20% organic active agent by weight. In any embodiment, the bolus article is configured to disintegrate in a stomach, for example a reticulo rumen, of the animal over a period of at least 1, 2, 3, 4, 5, 10, 15, 20, 25, 30, 35, 40, 45, 50, 55 or 60 days, for example 1 to 150 days, 10 to 120 days, 30 to 90 days, 40 to 80 days, 50 to 70 days, 1 to 5 days, 1 to 10 days, 10 to 20 days, 20 to 30 days, 30 to 40 days, 40 to 60 days, 50 to 70 days, or 50 to 60 days. In any embodiment, the bolus article is configured to release organic active agent in a stomach, for example a reticulo rumen of the animal at a rate of 10 to 500 mg, 10 to 200 mg, 50 to 150 mg, 80 to 120 mg, per day. In any embodiment, the bolus article has a specific gravity of 1 to 7, 1 to 6, 1 to 5, 1 to 4, 2 to 5, 2 to 4 g/cm3. In any embodiment, the bolus article has a surface area of 10 to 150, 10 to 100, 10 to 50, or 50 to 100 cm2. In any embodiment, the bolus article has a curved diameter of 10 to 50, 10 to 25, 25-50 mm.

As used herein, the term “carboxylate glass” refers to a glass formed when one or a mixture of metal carboxylates are heated to their melting temperature or above and allowed cool. In any embodiment, the carboxylate glass is selected from an acetate glass, a propanoate glass, a butanoate glass, and a mixed anion glass. The mixed anion glass may include carboxylates selected from an acetate, a propanoate, a butanoate and an octanoate. In any embodiment, the carboxylate glass comprises (or is formed from) a mixture of metal carboxylates, for example a mixture of metal acetates or a mixture of metal propanoates. Examples of carboxylates glasses are provided in Table 1 below. In any embodiment, the carboxylate glass comprises (or is formed from) at least 2, 3 or 4 metal carboxylates. In any embodiment, the cation of the metal carboxylate is selected from sodium, potassium, calcium and zinc, although other metals may be employed

TABLE 1

| | | Liquidus | Estimated | |
|---|---|---|---|---|
| | | temperature | duration of | Density |
| Glass composition ratio | | (º C.) | glass | g/cm3 |
| Acetate glass | | | | |
| Sodium acetate | | 140 | 2 hours | 1.48 |
| Potassium acetate | 1.1.1 | | | |
| Calcium acetate | | | | |
| Propanoate glass | | | | |
| Sodium propanoate | 1.1 | 180 | 8 hours | 1.32 |
| Calcium propanoate | | | | |
| Mixed anion glass | | | | |
| Sodium butanoate | | 160 | 2 weeks | 1.18 |
| Calcium propanoate | 1.2.2 | | | |
| Sodium octanoate | | | | |

As used herein the term “bromoform” refers to a brominated organic solvent obtainable from certain seaweed species, including species of Asparagopsis such as for example Asparagopsis taxiformis and/or Asparagopsis armata. In any embodiment, the bromoform is provided as a seaweed extract enriched in bromoform. The term “enriched in bromoform” means that bromoform constitutes at least 10%, 20%, 30%, 40% or 50% by weight of the seaweed extract. In any embodiment, bromoform constitutes at least 60%, 70% or 80% by weight of the seaweed extract. In any embodiment, the bromoform is a methanolic extract of seaweed. Methods of obtaining bromoform methanolic extracts from seaweed are described in WO2020124167 (methanolic extract method pages 36-37 and Table 2). In any embodiment, the bromoform is an oil extract of seaweed. Methods of obtaining bromoform oil extracts from seaweed are described in WO2020113279 (oil extract method of Example 1 pages 47 to 51). Additional methods of extracting bromoform from seaweed are described in WO2005/015983; U.S. Pat. No. 6,346,252; Algal Research, Volume 51, Pages 102065 (2020); J. Appl. Phycol., 28 (2016), pp. 3117-3126; Limnology and Oceanography, Issue: 5, Volume: 44, Pages 1348-1352. Jul. 13, 1999; Tetrahedron Letters, Issue: 7, Volume: 16, Pages: 473-476. 1975; Heliyon, Volume 4, Issue 11, November 2018, e00957; and Mar Ecol Prog Ser 306:87-101 (2006a). In any embodiment, the seaweed extract is lyophilized.

The invention also provides a method of forming a water-soluble glass bolus article. In one embodiment, the method comprises melting a metal carboxylate to form a liquid carboxylate glass, cooling the liquid carboxylate to provide a solid carboxylate glass, milling the solid carboxylate glass to provide carboxylate glass particles, mixing the carboxylate glass particles with bromoform, and sintering the mixture to form a sintered water-soluble glass bolus article. In another embodiment, the glass forming carboxylate(s) are mixed with the bromoform, melted to a liquid glass (typically at a temperature of less than 149° C.), and then cast into a glass bolus article. In any embodiment, the melting step is performed at a temperature of 50 to 300° C., 100 to 300° C., 100 to 250° C., 100 to 200° C., 150 to 300° C., 200 to 300° C., 150 to 250° C. or 200 to 250° C. In any embodiment, the sintering step is performed at a temperature of 20 to 250° C., 50 to 250° C., 100 to 250° C., 150 to 250° C., 50 to 200° C., 100 to 200° C. or 150 to 200° C. In any embodiment, the sintering step comprises an annealing step. In any embodiment, the annealing step is performed at a temperature of 20 to 200° C., 50 to 200° C., 100 to 200° C., 150 to 200° C., 50 to 1500° C., or 150 to 200° C. In any embodiment, the milling step is configured to provide carboxylate glass particles having an average particle size of 0.1 to 1 mm, 0.5 to 1 mm, 0.1 to 0.5 mm. In any embodiment, the mixing step comprises adding a binder to the carboxylate glass particles and organic active agent. In any embodiment, the mixture of carboxylate glass particles, organic active agent and optionally binder is granulated prior to the sintering step. Sintering or frittage is the process of compacting and forming a solid mass of material by heat and/or pressure without melting it to the point of liquefaction. It is described in German et al. (Encyclopedia of Materials: Science and Technology, 2001 and Sintering: from Empirical Observations to Scientific Principles, 2014. In another embodiment, a polymer may be used in a non-sintered bolus. In this case, the glass is prepared either as a carboxylate or phosphate glass, then milled to a particular size and mixed with bromoform and optionally a densifier, added to a mould, then the polymer is added and the materials pressed to solidify the mixture.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

A mixture of 1:1:1 sodium acetate, potassium acetate and calcium acetate is mixed together and heated to 140° C. until the mixture liquefies. The liquid glass is allowed to cool and is then milled and screened to an average particle size of 0.1 to 1 mm. Lyophilised seaweed extract is prepared according to the method of Example 1 of WO2020113279. The extract contains about 60 bromoform by weight. The lyophilized extract is added to the particulate glass in a weight ratio of 1 part seaweed extract to 6 parts particulate glass, mixed with any binding agent if applicable and pressed into a bolus of required dimensions. The pressed boluses are then sintered at a pre-determined temperature and annealed at a pre-determined temperature (obtained through analysis of the glass by differential scanning calorimetry).

### Example 2

The method of Example 1 is repeated except that the glass is formed from 1:1 mixture of sodium propanoate and calcium propanoate heated to 180° C. until the mixture liquefies.

### Example 3

The method of Example 1 is repeated except that the glass is formed from a mixture of 1:2:2 Sodium butanoate, Calcium propanoate and Sodium octanoate heated to 160° C. until the mixture liquefies.

### Example 4

15 g of lyophilised seaweed extract is prepared according to the method of Example 1 of WO2020113279. The extract contains about 10 g bromoform. A 2:2:2:1 mixture of sodium acetate, potassium acetate, calcium acetate and lyophilised seaweed extract is mixed together and heated to 140° C. until the mixture liquefies. The liquid glass is cast into glass articles having a diameter of 1.5 cm, length of 4 cm, and weight of 27 g.

### Example 5

A carboxylate or phosphate glass is prepared and milled to a specific particle size. This glass is mixed with the bromoform and placed into a mould. The polymer binder is added to the mould and then pressed to solidify the bolus. (polymer bonded glass bolus).

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A water soluble or water dispersible rumen bolus article suitable for oral administration to a reticulo rumen of a ruminant animal comprising a seaweed extract enriched in bromoform.

2. A water soluble or water dispersible rumen bolus article according to claim 1, in which the seaweed extract enriched in bromoform is a methanolic extract or an oil extract of seaweed.

3. A water soluble or water dispersible rumen bolus article according to claim 1 or 2, in which bromoform constitutes at least 60% of the seaweed extract by weight.

4. A water soluble or water dispersible rumen bolus article according to any preceding claim, in which the seaweed extract is lyophilised seaweed extract.

5. A water soluble or water dispersible rumen bolus article according to any preceding claim, in which the seaweed extract enriched in bromoform constitutes 1 to 20% by weight of the water soluble or water dispersible rumen bolus article.

6. A water soluble or water dispersible rumen bolus article according to claim 5, in which the seaweed extract enriched in bromoform constitutes 5 to 215% by weight of the water soluble or water dispersible rumen bolus article.

7. A water soluble or water dispersible rumen bolus article according to any preceding claim, comprising 5-15 g bromoform and configured to release 10-200 mg bromoform per day in the reticulo rumen of a ruminant animal.

8. A water soluble or water dispersible rumen bolus article according to any preceding claim comprising a water soluble matrix and bromoform distributed throughout the matrix, in which the water soluble matrix comprises a water soluble glass.

9. A water soluble or water dispersible rumen bolus article according to claim 8 in which the water soluble glass comprises a carboxylate glass.

10. A water soluble or water dispersible rumen bolus article according to claim 8 in which the carboxylate glass is formed from one or more metal carboxylates having a melting point of less than 300° C.

11. A water soluble or water dispersible rumen bolus article according to claim 8 in which the water soluble glass comprises a polymer bonded phosphate glass.

12. A water soluble or water dispersible rumen bolus article according to any of claims 1 to 11, for use in a method of administering bromoform to a ruminant animal by sustained release, the method comprising orally administering the rumen bolus article to the reticulo rumen of the animal wherein the bromoform is released from the rumen bolus article in the reticulo rumen of the animal over a sustained period of time.

13. A water soluble or water dispersible rumen bolus article according to any of claims 1 to 11, for use of claim 11, in which the rumen bolus article comprises 5-15 g bromoform, and is configured to release 10-200 mg bromoform per day in the reticulo rumen of the animal for a sustained period of 20-100 days.

14. A water soluble or water dispersible rumen bolus article according to any of claims 1 to 11, for use in a method of reducing methane production in a ruminant animal, the method comprising a step of administering the water-soluble or water dispersible rumen bolus article to the reticulo rumen of the animal, wherein the bromoform is released from the rumen bolus article in the reticulo rumen of the animal over a sustained period of time.
